(19) United States
(12) Patent Application Publication
(10) Pub. No.: US 2022/0072009 A1
(43) Pub. Date: Mar. 10, 2022

(54) SHORT-ACTING SELECTIVE GLUCOCORTICOID RECEPTOR MODULATORS

(71) Applicant: PANDA CONSULTING LLC, Concord, MA (US)

(72) Inventor: Ruth THIEROFF-EKERDT, Concord, MA (US)

(21) Appl. No.: 17/418,009

(22) PCT Filed: Dec. 23, 2019
(86) PCT No.: PCT/US2019/068296

§ 371 Date: Jun. 24, 2021

Related U.S. Application Data
(60) Provisional application No. 62/785,761, filed on Dec. 28, 2018.

(51) Int. Cl. A61K 31/573; A61K 9/48; A61K 9/00; A61K 9/50; A61P 25/22; A61K 31/569; A61K 31/58; A61K 31/567; A61K 31/56; A61K 31/352

(52) U.S. Cl. CPC A61K 31/573; A61K 9/4866; A61K 9/0004; A61K 9/5026; A61K 31/352; A61K 31/569; A61K 31/58; A61K 31/567; A61K 31/56; A61P 25/22

(57) ABSTRACT

Short-acting selective glucocorticoid receptor modulators for preventing, relieving or treating symptoms, disorders and diseases associated with acute stress or temporary hypercortisolism and compositions comprising immediate release and a plurality of delayed pulse releases of the modulators.

### CROSS-REFERENCE TO RELATED APPLICATION

This application claims the benefit of U.S. Provisional Patent Application No. 62/785,761, filed Dec. 28, 2018, which is hereby incorporated by reference in its entirety.

### FIELD OF THE INVENTION

The invention relates generally to the field of preventing, relieving and treating symptoms, disorders and diseases associated with acute stress or temporary hypercortisolism. More particularly, the invention relates to preventing, relieving and treating symptoms of stress-related academic and educational problems, adjustment disorders, insomnia disorders, and circadian rhythm disorders, by administration of immediate release, delayed release and/or pulsatile release compositions of Short-Acting Selective Glucocorticoid Receptor Modulators (“SASGRM(s)”), including, but not limited to, hydroxy-androsta-4,9(11)-dien-3-ones (e.g., RU-43044), 21-hydroxy-6,19-oxidoprogesterones, 16-hydroxy-11-(substituted phenyl)-estra-4,9-dienes (e.g., ORG 36410), 17-beta-carboxamides of dexamethasone, and Δ1-11-oxa-11-deoxycortisols.

### BACKGROUND OF THE INVENTION

Various publications, including patents, published applications, technical articles and scholarly articles are cited throughout the specification. Each of these cited publications is incorporated by reference herein, in its entirety and for all purposes.

All organisms must maintain a dynamic equilibrium, or homeostasis. Stress occurs when homeostasis is disturbed or perceived to be disturbed, triggering adaptive response to reestablish homeostasis.¹ One of the principal peripheral effectors of the stress system are glucocorticoids, the most important one in humans being cortisol.²

Cortisol is a pleiotropic steroidal hormone synthesized and secreted by the adrenal gland. It is one of the main mediators of the stress response³, as well as a main mediator of the sleep-wake cycle, and acts upon many body systems and organs including the cardiovascular system, energy metabolism, immunity, and the brain, where the cortisol level affects cognition, memory, the sleep-wake system and other brain functions and neurotransmitters.⁴

Cortisol exerts its effects by binding to the Glucocorticoid Receptor (“GR”) and Mineralocorticoid Receptor (“MR”), both members of the nuclear hormone superfamily. The GR functions as ligand-activated transcription factor that activates or represses target gene transcription through various mechanisms, including binding to GR-responsive elements as a homo- or heterodimer, through protein-protein interactions with other transcription factors, or by sequestering other transcription factors to inhibit their binding to DNA⁵.

Cortisol levels have a circadian rhythm driven by a pacemaker in the suprachiasmatic nucleus of the hypothalamus, with a daily peak around the time of the sleep-wake transition early in the morning and minimal levels in the evening and early part of the night. In addition, cortisol levels are subject to negative feedback regulation through the hypothalamic-pituitary-adrenal (HPA) axis, with cortisol inhibiting the hormones that activate its synthesis and secretion, corticotropin-releasing hormone (CRH) in the hypothalamus and adrenocorticotropic hormone (ACTH) in the pituitary gland.⁴ The feedback loop results, in addition to a circadian rhythm of cortisol, in an ultradian pattern of cortisol secretion with near-hourly pulses of cortisol peaks.⁶ This pulsatile rhythm is important for the biological outcome of cortisol signaling. Last, local expression of the enzyme 11-β-hydroxysteroid-dehydrogenase (11β HSD) type 1 and 2 modulates local cortisol concentration and influences whether MR, GR, or both, are occupied by cortisol depending on the organ.⁷

Thus, the control of cortisol levels and its mode of action is very complex, and the outcome of its binding to the GR depends on local cortisol concentration, its pulsatility, expression levels of the GR, co-expression of MR, and the presence at the molecular level of tissue- and cell-specific co-factors and co-repressors. This complexity greatly impedes the identification of selective SASGREMs that are active in vivo, show a good dissociation between desired activity in various organs or biologic systems and metabolic and immunosuppressive effects on the HPA axis.

Distress that is out of proportion with expected reactions to the stressor is defined as adjustment disorder⁸ Symptoms are clinically significant, causing marked distress and impairment in functioning. This distress and impairment are related to the stressor.

One of the most common adjustment disorders with functional impairment under stress is academic disruption. For example, many students cannot cope with the stress caused by either academic workload or an upcoming exam. It has been shown in a validated experimental setting for academic and social stress (Trier Social Stress Test) that cortisol rises in such situations.⁹ Timing of the cortisol rise can impair memory consolidation and memory retrieval, contributing beyond acute anxiety to academic disruption).¹⁰.

Hyperarousal resulting from a stressful day acutely delays sleep onset and disrupts sleep maintenance. The stress-associated insomnia leads to reduced performance and links stress and insomnia in a vicious cycle. Sleep is regulated by the interaction of two oscillatory processes, sleep homeostasis and a circadian pacemaker.¹¹ The circadian sleep-wake cycle is regulated by a circadian pacemaker located in suprachiasmatic nuclei of the anterior hypothalamus, and its timing is synchronized (entrained) to light/dark and social activities. Sleep phases are closely associated with the cortisol secretion pattern reflecting the diurnal variation of the hypothalamo-pituitary-adrenal (HPA) axis activity. Cortisol has a peak shortly after waking (cortisol awakening response) and then decreases during the day reaching a minimum (nadir) around midnight. The changes in cortisol coincide with sleep cycles, with low levels of cortisol during the first half of the night dominated by Slow wave sleep (SWS), and increasing cortisol during the second half of the night dominated by Rapid eye movement (REM) sleep.⁴

Some sleep disorders are associated with hypercortisolism, disturbed timing and amplitude of cortisol secretion, and hyperactivity of the HPA axis. An insomnia phenotype with subjective chronic primary insomnia and objective short sleep duration was shown to be associated with increased cortisol plasma levels in contrast to a phenotype with normal objective sleep duration.¹²

Changes in sleep patterns in healthy aging comprise decreased SWS, early awakening and increased number of awakening and have been associated with HPA axis hyperactivity. Cortisol plasma levels also were found to be significantly increased after traveling through time zones or experimental abrupt time shifts, along with profound changes in the temporal pattern of cortisol secretion.¹³ Chronic disruption of the circadian rhythm was found to be associated with elevated salivary cortisol and temporal lobe atrophy.¹⁴

### SUMMARY OF THE INVENTION

The invention features methods of relieving, preventing, and treating symptoms, disorders and diseases associated with acute stress or temporary hypercortisolism, comprising administering to a subject in need thereof a Short-Acting Selective Glucocorticoid Receptor Modulator (“SASGRM”), including but not limited to a SASGRM compound selected from:

hydroxy-androsta-4,9(11)-dien-3-ones, for example compound RU-43044 of Formula I

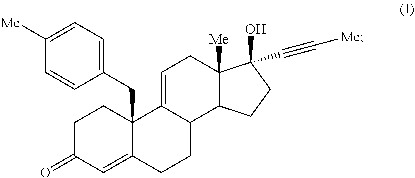 [SMILES: CC#C[C@]1(O)CCC2C3CCC4=CC(=O)CC[C@]4(Cc4ccc(C)cc4)C3=CC[C@@]21C]

21-hydroxy-6,19-oxidoprogesterones, for example a compound of Formula II

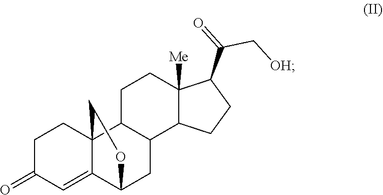 [SMILES: C[C@]12CCC3C(C[C@H]4OC[C@@]35CCC(=O)C=C45)C1CC[C@@H]2C(=O)CO]

16-hydroxy-11-(substituted phenyl)-estra-4,9-dienes, for example compound ORG 36410 of Formula III

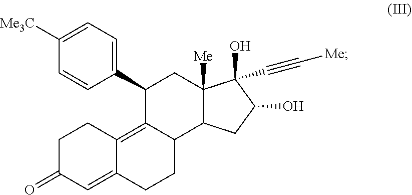

17-beta-carboxamides of dexamethasone, for example a compound of Formula IV

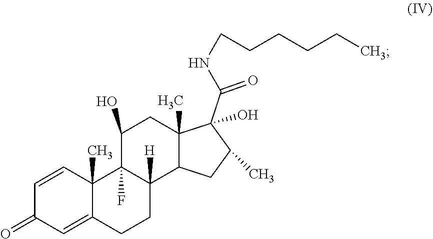

and
Δ1-11-oxa-11-deoxycortisols, for example a compound of Formula V

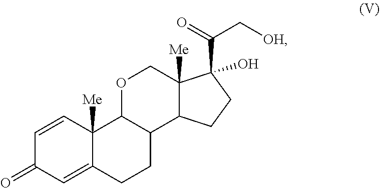 [SMILES: C[C@]12C=CC(=O)C=C1CCC1C2OC[C@@]2(C)C1CC[C@]2(O)C(=O)CO]

or a combination thereof and pharmaceutically acceptable salts and solvates thereof, in an amount effective to prevent, relieve or treat symptoms, disorders and diseases associated with acute stress or temporary hypercortisolism. States of acute stress or temporary hypercortisolism include stress-related academic and educational problems, adjustment disorders, insomnia disorders, and circadian rhythm disorders. Specifically, a distress that is out of proportion with expected reactions to the stressor is defined as an adjustment disorder. Symptoms are clinically significant, causing marked distress and impairment in functioning. This distress and impairment are related to the stressor.

Specifically, insomnia disorders associated with temporary hypercortisolism include stress-related acute insomnia, as well as certain forms of chronic insomnia, including chronic insomnia with objective short sleep duration. Circadian rhythm disorders include jet lag type and shift-work type circadian rhythm disorders.

The present inventions also features compositions comprising a plurality of particles comprising immediate release particles and/or delayed release particles comprising an amount of a SASGRM together with a pharmaceutically acceptable carrier thereof and optionally other therapeutic agents. The SASGRMs of the composition, include but are not limited to: hydroxy-androsta-4,9(11)-dien-3-ones, for example a compound of Formula I; 21-hydroxy-6,19-oxidoprogesterones, for example a compound of Formula II; 16-hydroxy-11-(substituted phenyl)-estra-4,9-dienes, for example a compound of Formula III; 17-beta-carboxamides of dexamethasone, for example a compound of Formula IV; and Δ1-11-oxa-11-deoxycortisols, for example a compound of Formula V and pharmaceutically acceptable salts and solvates thereof and any combination thereof. The composition may preferably comprise, for example, immediate release and/or delayed release particles or a combination thereof, comprising the SASGRMs of Formula I, Formula II, Formula III, Formula IV, or Formula V and pharmaceutically acceptable salts and solvates thereof and any combination thereof together with a pharmaceutically acceptable carrier thereof and optionally other therapeutic agents. The delayed release particles may comprise pulsatile release particles.

In some aspects, the composition comprises a population comprising immediate release particles, and a first and second population comprising pulsatile release particles. The first population of pulsatile release particles releases the SASGRM following a lag period after the SASGRM is released from the immediate release particles, and the second population of pulsatile release particles releases the SASGRM following a lag period after the SASGRM is released from the first population of pulsatile release particles. In some aspects, the composition further comprises a third population of pulsatile release particles that release the SASGRM following a lag period after the SASGRM is released from the second population of pulsatile release particles. Each lag period may range from about 0.5 hours to about 3 hours, preferably from about 0.5 hours to about 2.5 hours and more preferably from about 1 hour to about 2 hours.

The invention also features compositions of the SASGRMs of each of Formula I, Formula II, Formula III, Formula IV or Formula V and pharmaceutically acceptable salts and solvates thereof and any combination thereof together with a pharmaceutically acceptable carrier thereof and optionally other therapeutic agents that result in a delayed release or pulsatile release pattern. The compositions include an immediate release population of SASGRM-containing particles and one or more delayed release or pulsatile populations of SASGRM-containing particles and pharmaceutically acceptable salts and solvates thereof and any combination thereof together with a pharmaceutically acceptable carrier thereof and optionally other therapeutic agents. The SASGRM in each particle may comprise Formula I, Formula II, Formula III, Formula IV, or Formula V and pharmaceutically acceptable salts and solvates thereof or any combination thereof. The compositions include the SASGRMs in an amount effective to prevent, relieve or treat symptoms, disorders and diseases associated with acute stress or temporary hypercortisolism.

A first population of pulsatile release particles releases the SASGRM following a lag period after the SASGRM is released from the immediate release particles. In some aspects, a second population of pulsatile release particles releases the SASGRM following a lag period after the SASGRM is released from the first population of pulsatile release particles. In some aspects, a third population of pulsatile release particles releases the SASGRM following a lag period after the SASGRM is released from the second population of pulsatile release particles. Each lag period may range from about 0.5 hour to about 24 hours, preferably 1 hour to about 18 hours. Each lag period may range from about 1 hour to about 6 hours, preferably about 1 hour to about 5 hours. Each lag period may range from about 0.5 hour to about 3 hours. Each lag period may range from about 0.5 hours to about 2.5 hours. Each lag period may range from about 1 hour to about 2 hours. Thus, the composition may comprise one, two, three, or more populations of the SASGRM pulsatile release particles.

The immediate release, delayed release and/or pulsatile release particles may comprise a coating. The coating may comprise a water soluble polymer. The water soluble polymer may comprise methylcellulose, hydroxyethyl cellulose, hydroxypropyl cellulose, hydroxypropyl methylcellulose, methacrylic acid copolymers, cellulose acetate phthalate, polyvinylpyrrolidone, polyvinylpyrrolidone/vinyl acetate copolymer, Eudragit® L, Eudragit® S, polyvinyl alcohol, polyethylene glycol, polyethylene oxide, hyaluronic acid, alginate, carrageenan, gelatin, or any combination thereof. The coating may comprise a water insoluble polymer. The water insoluble polymer may comprise polyvinyl acetate, cellulose acetate, methyl cellulose, ethylcellulose, cellulose acetate butyrate, cellulose acetate propionate, noncrystalline cellulose, polyethylenes, chitosan, polyvinyl alcohol, polyacrylates, methacrylates, Eudragit® RS, Eudragit® RL, Eudragit® RS30D, Eudragit® RL30D, a Eudragit® NE30D, Methocel® K100M, Methocel® K15M or any combination thereof.

The coating may optionally include a plasticizer. The plasticizer may comprise triethyl citrate. The coating may optionally include a glidant. The glidant may comprise talc. The coating may optionally include an osmotic agent.

The immediate release, delayed release and/or pulsatile release particles may comprise a seal coating.

The immediate release, delayed release and pulsatile release particles may be comprised in a dosage form comprising all particle types, including all delayed release and/or all pulsatile release populations. The immediate, delayed release and pulsatile release particles may be in separate dosage forms. Each delayed release and/or pulsatile release population may be in a separate dosage form. The dosage forms may comprise a tablet, a caplet or a capsule, such as a gelatin capsule.

### BRIEF DESCRIPTION OF THE DRAWINGS

FIG. 1 is a graph of the results of Example 2, Experiment 1, from Table 2 with respect to the number of mice entries;

FIG. 2 is a graph of the results of Example 2, Experiment 1, from Table 2 with respect to the time spent in the open areas;

FIG. 3 is a graph of the results of Example 2, Experiment 2, from Table 4 with respect to the number of mice entries;

FIG. 4 is a graph of the results of Example 2, Experiment 2 from Table 4 with respect to the time spent in the open areas;

FIG. 5 is a graph of the results of Example 2, Experiment 3, from Table 6 with respect to the number of mice entries; and,

FIG. 6 is a graph of the results of Example 2, Experiment 3 from Table 6 with respect to the time spent in the open areas.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

The term “immediate release” is known in the art and includes compositions and active ingredients that are formulated, preferably as oral dosage forms, to release the composition or active ingredient immediately or as quickly as possible after administration without delaying or prolonging the dissolution or absorption of the composition or active ingredient.

The term “delayed release” is known in the art and includes compositions and active ingredients that are formulated, preferably as oral dosage forms, to release the composition or active ingredient only at some time point after the initial administration of the composition or active ingredient other than immediately after administration.

The term “pulsatile release” is a form of delayed release known in the art and includes compositions and active ingredients formulated, preferably as oral dosage forms, for rapid and transient release of the composition or active ingredient within a short time period immediately after a predetermined off-released period, i.e., lag time, or by a mechanism of delivering the composition or active ingredient rapidly and completely after a lag time, i.e., a period of no release of the composition or active ingredient. The term also includes controlled-release dosage forms, preferably oral dosage forms, with a pulsatile component, producing at least one timed pulse after an initial immediate release pulse, each pulse being characterized by a rapid and substantially complete release of the active ingredient. Such systems are known in the art as pulsatile compound delivery systems (PDDS), time-controlled systems, or sigmoidal release systems and include commercial systems such as Pulsincap™, Diffucap®, CODAS®, OROS®, IPDAS®, GEOCLOCK® and Uniphyl®.

Hydroxy-androsta-4,9(11)-dien-3-ones comprise RU-43044 ((10R,13S,17S)-17-hydroxy-13-methyl-10-[(4-methylphenyl)methyl]-17-prop-1-ynyl-2,6,7,8,12,14,15,16-octahydro-1H-cyclopenta[a]phenanthrene-3-one also know as 17-beta-hydroxy-17-alpha-19-(4-methylphenyl)androsta-4,9(11)-dien-3-one) of Formula I:

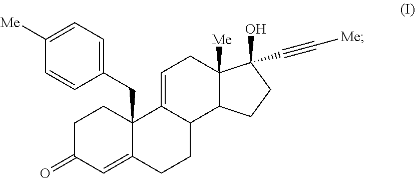 [SMILES: CC#C[C@]1(O)CCC2C3CCC4=CC(=O)CC[C@]4(Cc4ccc(C)cc4)C3=CC[C@@]21C]

21-hydroxy-6,19-oxidoprogesterones comprise a compound of Formula II:

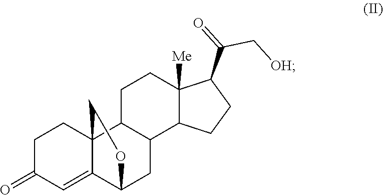 [SMILES: C[C@]12CCC3C(C[C@H]4OC[C@@]35CCC(=O)C=C45)C1CC[C@@H]2C(=O)CO]

16-hydroxy-11-(substituted phenyl)-estra-4,9-dienes comprise ORG 36410 ((11-beta, 16-alpha, 17-beta)-11-(4-isopropylphenyl)-16,17-dihydroxy-17-(1-propynyl)estra-4,9-dien-3-one) of Formula III:

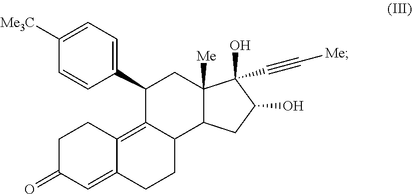

17-beta-carboxamides of dexamethasone comprise a compound of Formula IV:

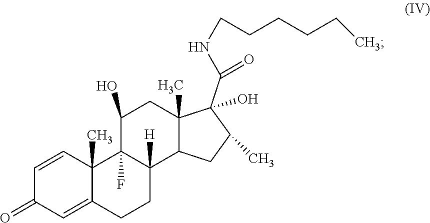

and,
Δ1-11-oxa-11-deoxycortisols comprise a compound of Formula V:

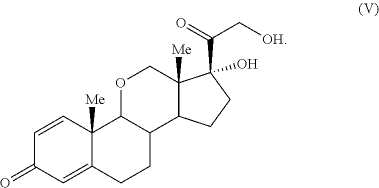 [SMILES: C[C@]12C=CC(=O)C=C1CCC1C2OC[C@@]2(C)C1CC[C@]2(O)C(=O)CO]

The terms subject and patient are used interchangeably. A subject may be any organism, including mammals such as farm animals (e.g., horse, cow, sheep, pig), laboratory animals (e.g., mouse, rat, rabbit), companion animals (e.g., dog, cat), and non-human primates (e.g., new world monkey and old world monkey). In preferred aspects, the subject is a human.

Symptoms and diseases associated with acute stress or temporary hypercortisolism include stress-related academic and educational problems, adjustment disorders, insomnia disorders, and circadian rhythm disorders. Non-limiting examples of academic and educational problems are failing school examinations, receiving failing marks or grades or underachievement (below what would be expected given the individual's intellectual capacity). Non-limiting examples of adjustment disorders include adjustment disorders with anxiety, characterized by nervousness, worry, jitteriness, or separation anxiety. Non-limiting examples of insomnia and circadian rhythm disorders include short-term sleep disruptions caused by time zone changes (e.g., jet lag) and shift work, as well as those caused by stressful events (e.g., family, financial, or work-related distress), and general changes in the normal sleep routine and/or environment, as well as certain forms of chronic insomnia such as chronic insomnia with objective short sleep duration associated with temporary hypercortisolism.

The SASGRMs of the present invention, including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), 17-beta-carboxamides of dexamethasone (e.g., Formula IV), and Δ1-11-oxa-11-deoxycortisols (e.g., Formula V) when administered as successive, appropriately timed and tailored doses, prevent, relieve, treat and/or beneficially affect symptoms, disorders and diseases associated with acute stress or temporary hypercortisolism. The SASGRMs of the present invention, including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) have limited metabolic stability, cross the blood-brain barrier, have selective antagonistic activity at the glucocorticoid receptor, and do not substantially change cortisol levels in blood after oral administration (i.e., do not disrupt the HPA axis), which properties can be advantageously utilized in terms of beneficially affecting a single or intermittent multiple dose-based regimen ideal for preventing, relieving and treating symptoms, disorders and diseases associated with acute stress or temporary hypercortisolism. Accordingly, the invention features methods for relieving, preventing, and treating symptoms, disorders and diseases associated with acute stress or temporary hypercortisolism with the SASGRMs of the present invention, including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) or any combination thereof and pharmaceutically acceptable salts and solvates thereof, as well as compositions of such SASGRMs, including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) or any combination thereof and pharmaceutically acceptable salts and solvates thereof, and the use of such compositions for preventing, relieving and treating symptoms and diseases associated with acute stress or temporary hypercortisolism. Preferably, the compositions are pharmaceutical compositions comprising delayed release and/or pulsatile release particles of the SASGRMs.

The present invention also provides the use of the SASGRMs of the present invention including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) or any combination thereof and pharmaceutically acceptable salts and solvates thereof, for the manufacture of a medicament for relieving, preventing, and treating symptoms, disorders and diseases associated with acute stress or temporary hypercortisolism. Preferably, the medicament is a medicament comprising delayed release and/or pulsatile release particles of the SASGRMs.

In general, methods for relieving, preventing, and treating symptoms, disorders and diseases associated with acute stress or temporary hypercortisolism comprise administering to a subject in need thereof an effective amount of a SASGRM, including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) or any combination thereof and pharmaceutically acceptable salts and solvates thereof, as part of a regimen for counteracting, preventing, relieving or treating symptoms, disorders and diseases associated with acute stress or temporary hypercortisolism. Other SASGRMs that have limited metabolic stability, cross the blood-brain barrier, have selective antagonistic activity at the glucocorticoid receptor and do not substantially enhance cortisol levels in the blood may also be used as part of a regimen for counteracting, preventing, relieving or treating symptoms, disorders and diseases associated with acute stress or temporary hypercortisolism.

The amount of the SASGRM of the present invention including hydroxy-androsta-4,9(11)-dien-3-ones (e.g., Formula I), 21-hydroxy-6,19-oxidoprogesterones (e.g., Formula II), 16-hydroxy-11-(substituted phenyl)-estra-4,9-dienes (e.g., Formula III), Δ1-11-oxa-11-deoxycortisols (e.g., Formula IV), and Δ1-11-oxa-11-deoxycortisols (e.g., Formula V) or any combination thereof and pharmaceutically acceptable salts and solvates thereof, which is required for counteracting, preventing, relieving or treating symptoms, disorders and diseases associated with acute stress or temporary hypercortisolism will, of course, vary with the particular compound, the route of administration, the age and condition of the recipient, and the particular symptom, disorder or disease being counteracted, prevented, relieved or treated.

A suitable daily dose for any of the above-mentioned symptoms, disorders or diseases will be in the range of 0.01 to 100 mg per kilogram body weight of the recipient (e.g., a human) per day, preferably in the range of 0.01 to 30 mg per kilogram body weight per day and most preferably in the range of 0.1 to 30 mg per kilogram, 0.1 to 20 mg per kilogram, 1 to 30 mg per kilogram, 1 to 10 mg per kilogram, 1 to 3 mg per kilogram body weight per day. The dose may be 1, 3, 10, 30 or 100 mg per kilogram body weight per day. The desired dose may be presented as one, two, three, four, five or more sub-doses administered at appropriate intervals throughout the day. Most preferably, the dose is given just before the recipient goes to sleep.

A SASGRM or combination of such may, for example, be formulated as a composition, preferably as a pharmaceutical composition, in immediate release, delayed release and/or pulsatile release particles, for example, as described or exemplified herein. A SASGRM or combination of such is preferably administered in a way that it may cross the blood brain barrier of the subject, and antagonize the glucocorticoid receptor in brain cells such that academic performance and adjustment to stressful situations increases, and/or sleep may be induced and/or maintained after acute stress episodes or time zone travel, or shift work. The subject is preferably a human, and preferably is in need of preventing, relieving or treating symptoms of acute stress in an academic or educations environment induced by an exam situation, public speaking, or similar social situations requiring adjustment to stress, as well as preventing, relieving or treating insomnia and circadian rhythm disorders associated with temporary hypercortisolism.

As the SASGRMs with selective antagonistic activity at the glucocorticoid receptors are intended to be targeted to the cells of the brain, the agents preferably freely pass the blood brain barrier. The SASGRMs preferably are administered according to any methodology or route that is suitable for allowing the SASGRMs to reach their targeted cells. Administration may be passive or guided. Administration is preferably oral. In some aspects, administration may be non-oral administration, for example, rectal, vaginal, intranasal, topical (including, transdermal, buccal and sublingual) or parental administration of the SASGRMs. The compositions may be prepared by any methods well known in the art of pharmacy, for example, as described in the examples herein or using methods such as those described in the latest edition of Remington's Pharmaceutical Sciences (see especially, Pharmaceutical Preparations and their Manufacture).

It is preferred that the SASGRMs be delivered in appropriately delayed release or timed pulses either alone or in combination with immediate delivery of one or more SASGRMs. When delivered according to timed pulses, the short half-life of the SASGRMs results in true pulses of pharmacologic activity rather than a sustained pharmacological activity as seen, for example, with mifepristone. The discrete pulses of pharmacological activity of the SASGRMs may be synchronized with the ultradian pattern of cortisol levels found in healthy subjects, thus minimizing hypothalamic-pituitary-adrenal (HPA) axis dis-inhibition by cortisol. Also, the circadian rhythm of cortisol secretion, especially the cortisol peak in the morning known as awakening response, is maintained, therefore preserving the ability of the subject to adjust adequately to normal stress.

It is preferred that the release pattern of the SASGRMs for relieving, preventing, and treating symptoms, disorders and diseases associated with acute stress or temporary hypercortisolism comprises a single release from an immediate release composition preferably in combination with one or more delayed or pulsatile releases over a period of several hours, beginning with an immediate release and followed by one or more delayed or pulsatile releases following a lag time between each release over a total period of 0.5-24 hours, preferably 0.5-18 hours, more preferably 0.5-5 hours, more preferably 0.5-3 hours. The delayed or pulsatile releases preferably follow sleep onset. Any suitable number of pulses may be included with the SASGRM pulsatile compound delivery system, including two, three, four, or five pulses. Including the immediate release (a first pulse), the pulsatile compound delivery system may further include a second pulse following a lag period after the first pulse, and optionally, a third pulse following a lag period after the second pulse, and optionally, a fourth pulse following a lag period after the third pulse. In some preferred aspects, the pulsatile compound delivery system includes two total pulses. In some preferred aspects, the pulsatile compound delivery system includes three total pulses. In some preferred aspects, the pulsatile compound delivery system includes four total pulses.

The lag period includes the time between pulse delivery of the SASGRM from each population of the SASGRM-containing particles. It is preferred that the lag period between pulses is from about 0.5 hours to about 24 hours, preferably about 0.5 to about 5 hours, preferably 0.5 hours to about 3 hours, more preferably from about 0.5-1 hour, and more preferably to about 1-3 hours, and more preferably from about 1 hour to about 2 hours. The lag period between pulses may be about 0.5 hour, 1 hour, about 1.5 hours, about 1.75 hours, about 2 hours, about 2.25 hours, about 2.5 hours, about 2.75 hours, about 3 hours, about 3.5 hours, about 4 hours, about 4.5 hours, about 5 hours, about 5.5 hours, about 6 hours or about 18 hours.

Thus, the SASGRM is preferably delivered according to two to four distinct compound-containing fractions, which comprise a fast/immediate release fraction, a medium release fraction, releasing the SASGRM after a lag period, a medium to slow release fraction releasing the SASGRM after a second lag period, and a slow release fraction releasing the SASGRM after a third lag period. The last release occurs preferably about 5 to about 9 hours following the immediate release, more preferably about 5 hours or about 6 hours following the immediate release. The dose of the intermediate and last releases may be lower than the dose of the preceding releases.

The SASGRMs are preferably delivered according to an immediate release system, and more preferably according to a delayed or pulsatile release system, optionally in combination with an immediate release system, based on particle fractions of an overall compound delivery form. For example, two to four populations of SASGRM-containing particles may be prepared as a larger dosage form, with each population of particles designed to release the SASGRM according to a desired time frame. The SASGRM-containing particles may thus be prepared as a tablet or caplet, for example, or may be prepared as a capsule. The delayed or pulsatile release system may also comprise separately administered SASGRM-containing particle populations, for example, an immediate release tablet, caplet, or capsule, and one or more separate delayed or pulsatile release tablets, caplets, or capsules.

The SASGRM-containing particles may comprise particles, pellets, microparticles, nanoparticles, minitablets, beads, granules, or mixtures thereof. When formulated as a tablet or caplet delivery form, the particles may be compressed together. When formulated as a capsule delivery form, the particles may be filled into the capsule. Suitable delivery forms other than a tablet, caplet, or capsule may be used.

The particles may comprise an inert core, for example, a sugar seed onto which the SASGRM is coated. The particles may, in addition to the SASGRM, include one or more excipients and/or one or more coatings. Common excipients are known in the art, and include but are not limited to solubility enhancers, disintegrants, lubricants, binders, glidants, taste-masking or flavor agents, sweeteners, dyes, and other known excipients. The coatings may help maintain the particle form as well as affect the release rate and/or release location. The coatings preferably comprise water soluble and/or water insoluble polymers or copolymers, and may be pH dependent or pH independent. In some aspects, the particles comprise a sealing layer, which may encase the SASGRM-containing core and/or the delayed or pulsatile release-coated particle. For the immediate release population of particles, a sealing layer may be used in lieu of a coating layer.

Suitable water soluble polymers include, but are not limited to, methylcellulose, hydroxyethyl cellulose, hydroxypropyl cellulose, hydroxypropyl methylcellulose, cellulose acetate phthalate, polyvinylpyrrolidone, polyvinylpyrrolidone/vinyl acetate copolymer, methacrylic acid copolymers such as Eudragit® L and S, polyvinyl alcohol, polyethylene glycol, polyethylene oxide, hyaluronic acid, alginate, carrageenan, gelatin, and any combination thereof.

Suitable water insoluble polymers include, but are not limited to, polyvinyl acetate, cellulose acetate, methyl cellulose, ethylcellulose, cellulose acetate butyrate, cellulose acetate propionate, noncrystalline cellulose, polyethylenes, chitosan, polyvinyl alcohol, polyacrylates, methacrylates, Eudragit® RS, RL, RS30D, RL30D, and NE30D and other Eudragit® brand polymers, as well as Methocel® K100M and K15M and other Methocel® brand polymers, and any combination thereof.

In some aspects, a plasticizer may be included in the coating. Suitable plasticizers include, but are not limited to, glycerol, glycerin, triacetin, gylcerol triacetate, glycerin sorbitol, acetate esters, polyethylene glycol (PEG), triethyl citrate, acetyltriethyl citrate, tributyl citrate, diethyl phthalate, dibutyl phthalate, dihexyl phthalate, castor oil, rape seed oil, olive oil, sesame oil, monoacetylated and diacetylated glycerides, and any combination thereof.

In some aspects, a glidant may be included in the coating. Suitable glidants include, but are not limited to, magnesium stearate, calcium silicate, magnesium silicate, talc, glyceryl monostearate, and any combination thereof.

In some aspects, an osmotic agent may be included in the coating. Categories of suitable osmotic agents include, but are not limited to, salts, sugars, and acids. Suitable osmotic agents, include, but are not limited to magnesium chloride, magnesium sulfate, potassium chloride, potassium phosphate, sodium chloride, sodium phosphate, sodium sulfite, sodium citrate, citric acid, malic acid, fumaric acid, tartaric acid, mannitol, sorbitol, erythritol, dextrose, fructose, galactose, and any combination thereof.

The polymeric coating may be added to the SASGRM core to achieve from about 1% to about 40% weight gain to each particle. The polymeric coating may be added to achieve from about 2% to about 20%, from about 2% to about 15%, from about 2% to about 13%, from about 2% to about 12%, from about 2% to about 10%, from about 2% to about 9%, from about 2% to about 8%, from about 2% to about 7%, from about 2% to about 5%, from about 3% to about 30%, from about 3% to about 20%, from about 3% to about 15%, from about 3% to about 13%, from about 3% to about 12%, from about 3% to about 10%, from about 3% to about 9%, from about 3% to about 8%, from about 3% to about 7%, from about 3% to about 6%, from about 3% to about 5%, from about 5% to about 30%, from about 5% to about 25%, from about 5% to about 15%, from about 5% to about 12%, from about 5% to about 10%, from about 6% to about 15%, from about 6% to about 12%, from about 6% to about 10%, from about 8% to about 13%, from about 8% to about 12%, from about 8% to about 10%, from about 9% to about 12%, from about 9% to about 13%, from about 9% to about 11%, from about 10% to about 40%, from about 10% to about 20%, from about 10% to about 15%, from about 15% to about 25%, from about 15% to about 20%, from about 20% to about 30%, from about 20% to about 25%, from about 25% to about 40%, from about 25% to about 30%, or from about 30% to about 35% weight gain to each particle. Particles intended to comprise the second, third, or fourth pulsatile release may comprise increasingly more weight gain, with the thicker polymeric coatings allowing for a longer release delay.

In some aspects, the immediate release particles comprise about 2%, about 3%, about 4%, about 5%, or about 6% weight gain from the polymeric coating. The second delayed or pulsatile release particles comprise about 5%, about 6%, about 7%, about 8%, or about 9% weight gain from the polymeric coating. The third and subsequent delayed or pulsatile release particles comprise about 8%, about 9%, about 10%, about 11%, or about 12% weight gain from the polymeric coating.

The following examples are provided to describe the invention in greater detail. They are intended to illustrate, not to limit, the invention.

### Example 1

### Metabolic Stability and Blood-Brain Barrier Passage of Formula I, Formula II, Formula III, Formula IV and Formula V

The metabolic stability of the compound of each of Formulae I-V is either unknown or has been known to be very short (e.g., in vitro data for Formula I with a reported half life of 0.33 h in rat liver microsomes and less than 0.2 h in rat hepatocytes; Formula IV with a reported conversion to unidentified metabolites in rat liver microsomes within 30 min). This lead to discontinuation of previous development of such shot-acting GR ligands in favor of more metabolically stable compounds.

The metabolic stability of the compound of each of Formula I, Formula II, Formula III, Formula IV and Formula V can be investigated. The compounds should have limited metabolic stability in vitro and in vivo as compared to mifepristone (RU-486, a progesterone receptor modulator that also exhibits anti-glucocorticoid properties).

In order to determine the metabolic stability, 10⁻³M stock solutions of Formula I, Formula II, Formula III, Formula IV and Formula V in DMSO are diluted to 10⁻⁶ M and incubated in pooled human and rat liver microsome preparations, respectively, in potassium phosphate buffer pH 7.4 at 37° C. for over 45 min, and samples taken at 0, 5, 15, 30, 45 and 60 min. Ketanserin or testosterone are used as a positive control. The reaction is stopped by de-proteination with an organic solvent and, after mixing and centrifugation, samples are analyzed by liquid chromatography-mass spectrometry (LC-MS)/MS. Formula I, Formula II, Formula III, Formula IV and Formula V should show a half-life in pooled human and rat microsomes, respectively, between 5 min and 1 hour.

Subsequently, the metabolic stability of Formula I, Formula II, Formula III, Formula IV and Formula V can be investigated in mice in vivo. Formula I, Formula II, Formula III, Formula IV and Formula V are suspended in 0.5% (w/v) carboxymethylcellulose, and a single oral dose of 30 and 100 mg/kg is administered to male CD-1 mice (28-36 g) per oral gavage, with n=3 per group. Mifepristone at a dose of 10 mg/kg, and vehicle controls are used as a reference. Multiple blood samples are obtained by tail bleeding at 0, 5, 15, 30 min and 1, 2, 4, 8 and 24 hours following administration, with whole blood collected in heparin-coated tubes. Plasma is obtained after centrifugation at 2000×g for 10 min, and samples are subjected to LC-MS/MS. Plasma half-life in the mice after oral gavage is between 30 and 90 min. Corticosterone is analyzed in different aliquots of the same samples and is not found to be significantly increased by Formula I, Formula II, Formula III, Formula IV, and Formula V up to a dose of 30 mg/kg.

Next, the interaction of Formula I, Formula II, Formula III, Formula IV, and Formula V with the blood brain barrier (BBB) is assessed. As part of this investigation, binding to rat and human plasma proteins is studied using equilibrium dialysis at pH 7.4 and 37° C. against phosphate-buffered saline with test compounds at 5 μM and warfarin (an anti-coagulant) as a reference compound. Aliquots are removed from the sample (donor) and buffer (receiver) chamber after equilibrium is reached, and subjected to LC-MS/MS. Formula I, Formula II, Formula III, Formula IV, and Formula V show plasma protein binding below 50% in both species. In contrast, plasma protein binding over 95% has been reported for mifepristone in rat and human serum. BBB penetration is assessed in vitro using confluent MDCK cell monolayers and dosing of 504 of test compounds on apical and basolateral side of the monolayer at pH 7.4 for 90 min. The apparent permeability in both directions, and the efflux ratio is determined after subjecting samples to LC-MS/MS analysis. Formula I, Formula II, Formula III, Formula IV, and Formula V have a moderate to high BBB penetration potential.

### Example 2

### Short-Acting Selective Glucocorticoid Receptor Modulators in a Mouse Model of Stress-Induced Anxiety

Formula I, Formula II, Formula III, Formula IV, and Formula V constitute SASGRMs of diverse chemical structure with a similar biological profile. They show selectivity for glucocorticoid receptor (GR) binding, selective GR antagonism in vitro, limited metabolic stability in vitro and in vivo, no increase in corticosterone levels after oral administration in vivo as indicator of HPA axis dis-inhibition in mice, low plasma protein binding and moderate to high BBB penetration potential.

Formula III (also referred to as PND-001) as a representative compound out of the class of SASGRMs was tested in a mouse model of stress-induced anxiety, the elevated plus maze test (EPM) (Experiment 1). The EPM relies on the inherent conflict between a rodent's tendency to explore a novel environment on one hand, and the stress of such exploration, resulting in avoidance of the aversive properties of the brightly lit open arms. Compounds that reduce anxiety specifically increase the frequency of entries into the open arms and time spent in the open arms.

Four to five week-old male CD-1 mice were group-housed (6 or 7 mice per cage) and maintained in a room with controlled temperature (21-22° C.) and a reversed light-dark cycle (12 h/12 h; lights on: 17:30-05:30; lights off: 05:30-17:30) with food and water available ad libitum.

Formula III and the vehicle (5% Dimethyl Sulfoxide (DMSO) and 95% Polyethylene Glycol 400 (PEG400)) were s.c. administrated 24 hours and 6 hours prior the EPM trial.

Diazepam was p.o. administrated 60 min prior the EPM trial as a positive control.

Formula III was tested in doses of 10, 30 and 100 mg/kg. Diazepam was p.o administrated 60 min prior the EPM trial and tested in a dose of 1 mg/kg. (see Table 1).

TABLE 1
Treatment schedule

| Gr. | | | | Dosage | Time |
|---|---|---|---|---|---|
| no | Description | n | route | volume | before trial |
| 1 | Vehicle | 12 | sc | 10 ml/kg | 24 h and 6 h |
| 2 | Diazepam (1 mg/kg) | 12 | po | 10 ml/kg | 60 min |
| 3 | Formula III at 10 mg/kg | 12 | sc | 10 ml/kg | 24 h and 6 h |
| 4 | Formula III at 30 mg/kg | 12 | sc | 10 ml/kg | 24 h and 6 h |
| 5 | Formula III at 100 mg/kg | 12 | sc | 10 ml/kg | 24 h and 6 h |

### Experimental Procedure

The mice were randomly assigned to one of the different experimental groups. Each animal was identified by its group name, cage number, series (day) of experiment, and a number (from 1 to 12) written with permanent ink on its tail.

The apparatus was a PVC maze covered with Plexiglas and subdivided into four equal exploratory arms (21×8 cm), which are all interconnected by a small platform (8×8 cm). The apparatus was placed 59 cm above the floor. Two arms are opened and two others are closed with wall (high: 21 cm).

After compound administration, mouse was placed on the platform opposite a closed arm. The number of entries and the time spent in each arm were recorded during a 5 min period. The animal was considered as entered in an arm when it places its four paws in the arm.

The apparatus was cleaned between each animal using alcohol (70°). Urine and feces were removed from the maze.

During the trials, animal handling and the visibility of the operator were minimized as much as possible.

Calculation and Statistical Analysis

Analysis of variance (ANOVA) was performed on the result data. Fisher's PLSD was used for pairwise comparisons and p value ≤0.05 were considered significant.

Results

Formula III-treated mice showed significant increase in the number of visit and the time spent into the open arms (Table 2).

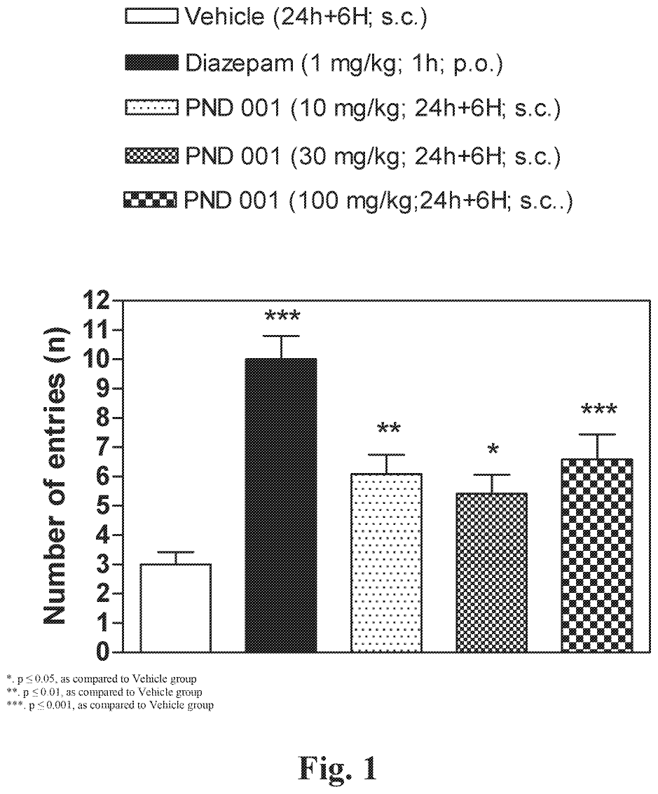

As shown in FIG. 1, Formula III was associated with a significant increase in the number of entries to the open arms. As compared to the performance of vehicle treated mice, the increase was 103%, 81%, and 119% for 10, 30 and 100 mg/kg of Formula III, respectively. FIG. 1 graph has a mean+/−s.e.m. of n=12 for all groups.

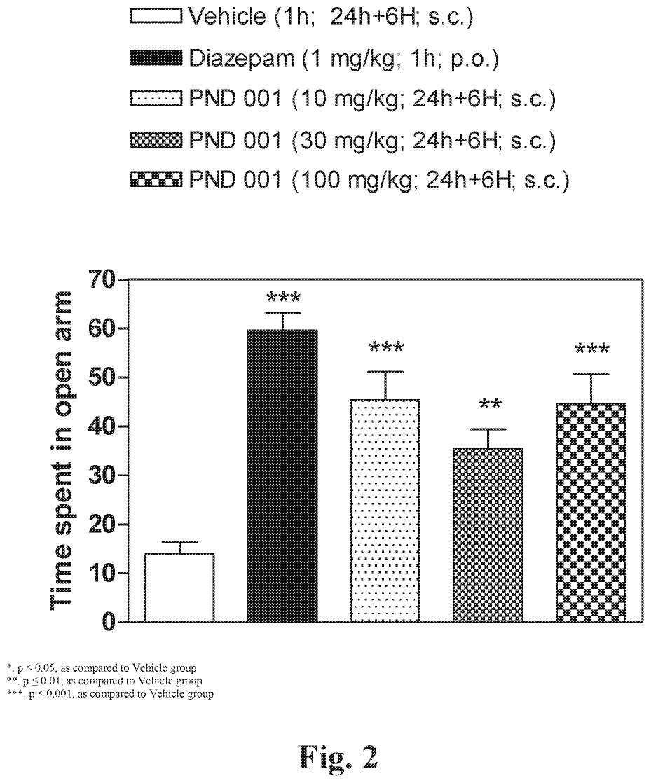

As shown in FIG. 2, Formula III also produced an increase in the time spent in the open arms. As compared to the vehicle treated mice, the increase was 224%, 154%, and 219% for 10, 30 and 100 mg/kg Formula III, respectively. FIG. 2 graph has a mean+/−s.e.m. of n=12 for all groups.

Diazepam treatment was associated with significant increase in both number of entries and time spent into the open arms. As compared to the performance of the vehicle treated mice, the increase was 233% and 327%, respectively.

TABLE 2
Behavioral performance during the EPM test

| | | | Number of entries | | Time spent in | |
|---|---|---|---|---|---|---|
| | | | in the open arm | | the open arm | |
| | | Mean | (n) | | (s) | |
| | | Weight | | Std. | | Std. |
| | n | (g) | Mean | Err. | Mean | Err. |
| Vehicle (24 + 6 h, sc) | 12 | 32.4 | 3.0 | 0.4 | 14.0 | 2.4 |
| Diazepam (1 mg/kg; 1 | 12 | 32.2 | 10.0 | 0.8 | 59.6 | 3.5 |
| h; po) | | | | | | |
| Formula III (10 mg/kg; | 12 | 32.5 | 6.1 | 0.7 | 45.3 | 5.8 |
| 24 + 6 h, sc) | | | | | | |
| Formula III (30 mg/kg; | 12 | 33.2 | 5.4 | 0.6 | 35.4 | 3.9 |
| 24 + 6 h, sc) | | | | | | |
| Formula III (100 | 12 | 32.7 | 6.6 | 0.9 | 44.6 | 6.1 |
| mg/kg; 24 + 6 h, sc) | | | | | | |

In the next experiment in the Elevated Plus Maze Test (Experiment 2; conducted as described under the same experimental procedures and analysis as described for Experiment 1), the efficacy of Formula III (also referred to as PND001) at lower doses was studied. In addition, the efficacy of Formula IV (also referred to as PND002) was studied at a dose that showed efficacy of Formula III in Experiment 1. Compounds or vehicle (5% Dimethyl Sulfoxide (DMSO) and 95% Polyethylene Glycol 400 (PEG400)) were administrated s.c. 24 hours and 6 hours prior the EPM trial (Table 3).

TABLE 3
Treatment schedule

| Gr. | | | | Dosage | Time |
|---|---|---|---|---|---|
| no | Description | n | route | volume | before trial |
| 1 | Vehicle | 12 | sc | 10 ml/kg | 24 h and 6 h |
| 2 | Diazepam (1 mg/kg) | 12 | po | 10 ml/kg | 60 min |
| 3 | Formula III at 1 mg/kg | 12 | sc | 10 ml/kg | 24 h and 6 h |
| 4 | Formula III at 3 mg/kg | 12 | sc | 10 ml/kg | 24 h and 6 h |
| 5 | Formula III at 10 mg/kg | 12 | sc | 10 ml/kg | 24 h and 6 h |
| 6 | Formula IV at 30 mg/kg | 12 | sc | 10 ml/kg | 24 h and 6 h |

Formula III (PND 001) was tested in doses of 1, 3 and 10 mg/kg. Formula IV (PND 002) was tested in doses of 30 mg/kg. Diazepam was p.o administrated 60 min prior the EPM trial as positive control and tested in a dose of 1 mg/kg.

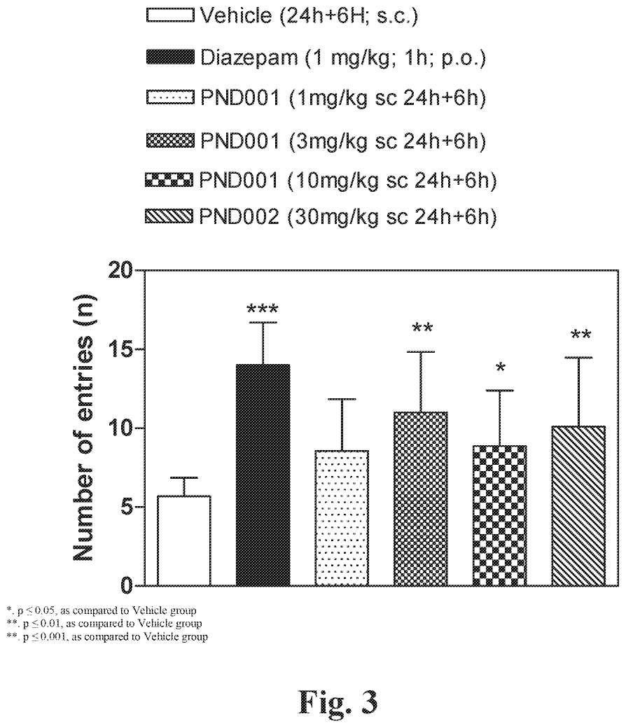

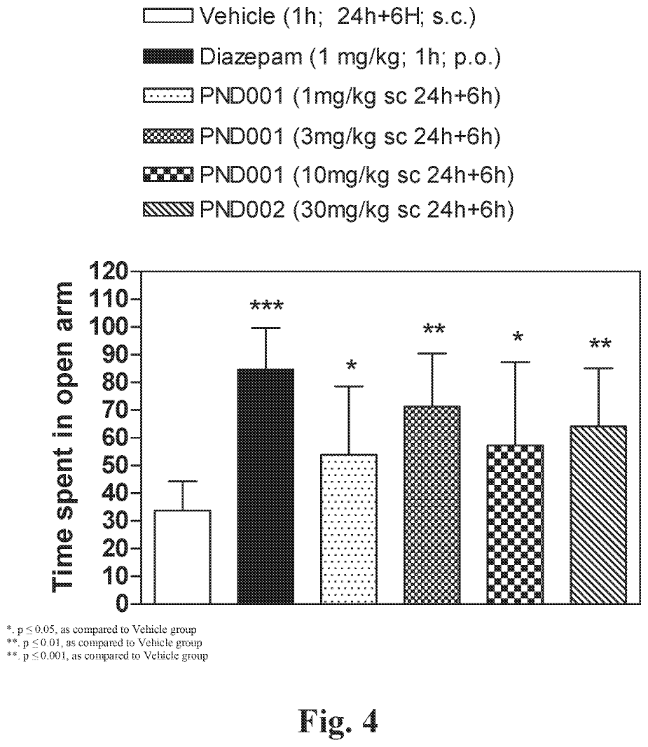

As shown in FIG. 3 and Table 4, PND 001 was associated with a significant increase in the number of entries to the open arms. FIG. 3 graph has a mean+/−SD of n=12 for all groups. As compared to the performance of vehicle treated mice, the increase was 50%, 93%, and 56% for 1, 3 and 10 mg/kg PND 001, respectively with the two higher doses reaching statistical significance. As shown in FIG. 4 and Table 4, PND 001 also produced an increase in the time spent in the open arms, which was statistically significant from vehicle at all doses. FIG. 4 graph has a mean+/−SD of n=12 for all groups. As compared to the vehicle treated mice, the increase was 60%, 112%, and 70% for 1, 3 and 10 mg/kg PND 001, respectively. PND 002 treatment was associated with significant increase in both number of entries and time spent into the open arms. As compared to the performance of the vehicle treated mice, the increase was 77% and 90%, respectively. Diazepam treatment as positive control was associated with significant increase in both number of entries and time spent into the open arms. As compared to the performance of the vehicle treated mice, the increase was 146% and 151%, respectively.

TABLE 4
Behavioral performance during the EPM test (Experiment 2)

| | | | Number of entries | | Time spent in | |
|---|---|---|---|---|---|---|
| | | | in the open arm | | the open arm | |
| | | Mean | (n) | | (s) | |
| | | Weight | | Std. | | Std. |
| | n | (g) | Mean | Err. | Mean | Err. |
| Vehicle (24 + 6 h, sc) | 12 | 32.4 | 5.7 | 0.4 | 33.7 | 3.4 |
| Diazepam (1 mg/kg; 1 | 12 | 31.6 | 14.0 | 0.9 | 84.6 | 4.8 |
| h; po) | | | | | | |
| PND 001 (1 mg/kg; | 12 | 32.3 | 8.6 | 1.1 | 53.9 | 8.2 |
| 24 + 6 h, sc) | | | | | | |
| PND 001 (3 mg/kg; | 12 | 31.7 | 11.0 | 1.3 | 71.3 | 6.4 |
| 24 + 6 h, sc) | | | | | | |
| PND 001 (10 mg/kg; | 12 | 31.8 | 8.9 | 1.2 | 57.3 | 10.6 |
| 24 + 6 h, sc) | | | | | | |
| PND 002 (30 mg/kg; | 12 | 32.2 | 10.1 | 1.5 | 64.1 | 7.0 |
| 24 + 6 h, sc) | | | | | | |

In a third experiment in the Elevated Plus Maze Test (Experiment 3), conducted as described under the same experimental procedures and analysis as described for Experiment 1, the effect of the dosing route and regimen was investigated. Formula III (also referred to as PND001) and the vehicle (5% Dimethyl Sulfoxide (DMSO) and 95% Polyethylene Glycol 400 (PEG400)) were administrated by gavage (p.o.). Diazepam was i.p. administrated 30 min prior the EPM trial as positive control.

PND 001 was tested in doses of 3 mg/kg which had the highest efficacy in Experiment 2, and administrated either twice at 6 h and 1 h before the EPM trial, or given only once at 1 h before the trial. Diazepam was i.p. administrated 30 min prior the EPM trial and tested in a dose of 1 mg/kg. (see Table 5).

TABLE 5
Treatment schedule (Experiment 3)

| Gr. | | | | Dosage | Time | |
|---|---|---|---|---|---|---|
| no | Description | n | route | volume | before trial | |
| 1 | Vehicle | 8 | po | 10 ml/kg | 6 h and 1 h | |
| 2 | Diazepam (1 mg/kg) | 8 | ip | 10 ml/kg | 30 | min |
| 3 | PND 001 at 3 mg/kg | 8 | po | 10 ml/kg | 1 | h* |

| | | | | | |
|---|---|---|---|---|---|
| 4 | PND 001 at 3 mg/kg | 8 | po | 10 ml/kg | 6 h and 1 h |

*Note that Group 3 received a vehicle dosing 6 h before the trial to obtain identical number of dosing as for group 1 and 4.

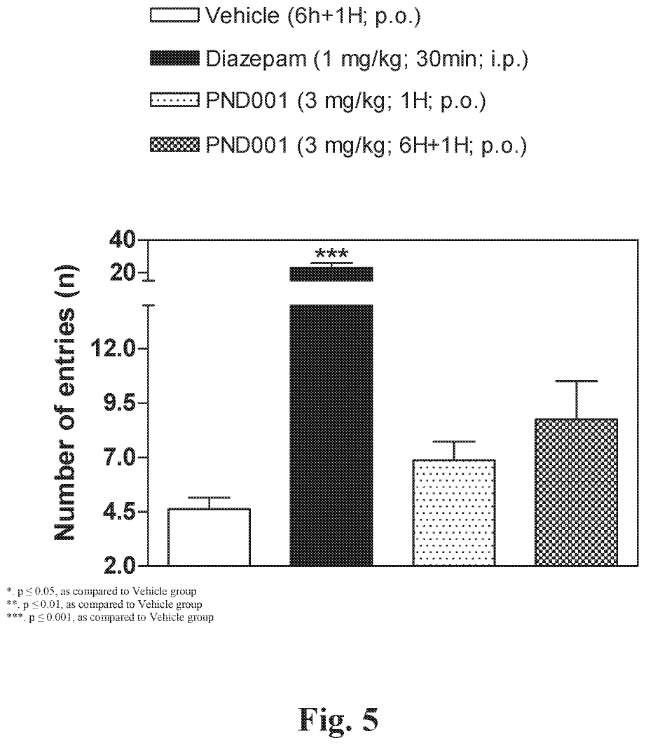

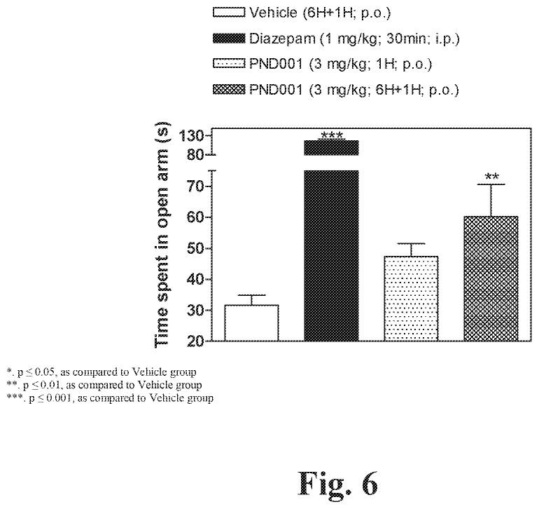

As shown in Table 6 and FIG. 6, PND 001 produced an increase in the time spent in the open arms. FIG. 6 graph has a mean+/−SEM of n=8 for all groups. As compared to the vehicle treated mice, the increase was 50% and 91% for 1 h pretreatment, and 1 h and 6 h pretreatment with PND 001, respectively. Only the pretreatment at 1 h and 6 h reached statistical significance. Pretreatment at 1 h and 6 h, but not pretreatment at 1 h only, showed an increase in the number of entries. As shown in Table 6 and FIG. 5, the number of entries approximately doubled as seen in Experiment 2 at the same dose with an s.c. pretreatment 24 h and 6 hours which however did not reach statistical significance over vehicle in this experiment. FIG. 5 graph has a mean+/−SEM of n=8 for all groups.

Diazepam treatment as positive control was associated with significant increase in both number of entries and time spent into the open arms. As compared to the performance of the vehicle treated mice, the increase was 400% and 270%, respectively.

TABLE 6
Behavioral performance during the EPM test (Experiment 3)

| | | | Number of entries | | Time spent in | |
|---|---|---|---|---|---|---|
| | | | in the open arm | | the open arm | |
| | | Mean | (n) | | (s) | |
| | | Weight | | Std. | | Std. |
| | n | (g) | Mean | Err. | Mean | Err. |
| Vehicle (6 + 1 h, po) | 8 | 31.2 | 4.6 | 0.5 | 31.6 | 3.3 |
| Diazepam (1 mg/kg; | 8 | 30.5 | 23.2 | 2.8 | 116.9 | 4.8 |
| 30 min; ip) | | | | | | |
| PND 001 (3 mg/kg; 1 | 8 | 33.3 | 6.9 | 0.9 | 47.4 | 4.1 |
| h, po) | | | | | | |
| PND 001 (3 mg/kg; | 8 | 33.6 | 8.8 | 1.8 | 60.2 | 10.4 |
| 6 + 1 h, po) | | | | | | |

Based on the results for Formula III and Formula IV in a mouse model of anxiety reflective of adjustment to stress, compounds of Formula I, Formula II, and Formula V and SASGREMs with a similar biological profile (specificity for GR, pure GR antagonistic activity in CNS short plasma half life and penetration of the BBB), formulated in appropriate pharmaceutical dosage forms, may be used as beneficial medicaments for preventing, relieving or treating symptoms, disorders and diseases associated with acute stress or temporary hypercortisolism, specifically adjustment (acute) insomnia, chronic insomnia associated with hypercortisolism, and circadian rhythm disorders of the jet lag type and shift work type.

Suitable pharmaceutical dosage forms for the intended use of SASGREMs such as Formula I, Formula II, Formula III, Formula IV, and Formula V in stress disorders, specifically sleep disorders associated with hypercortisolism, include orally-administered controlled-release dosage forms with a delayed release or pulsatile release component, producing at least one timed pulse after an initial immediate release pulse, each pulse being characterized by a rapid and substantially complete release of the active ingredient. Such systems are known as delayed release systems, pulsatile compound delivery systems (PDDS), time-controlled systems, or sigmoidal release systems.

### Example 3

### Preparation of Coated Pellets for Immediate Release (of Micronized RU 43044 (Formula I))

The particle size of RU 43044 (Formula I), for example, can be significantly reduced by using specific milling techniques like jet stream milling in a steel chamber under nitrogen pressure. The micronization occurs when the RU 43044 (Formula I) powder is fed at a velocity of about 50 m/s into the milling chamber and the faster particles, accelerated by a series of jet nozzles to a speed of about 300 m/s collide with the incoming slower particles. The particle size distribution (by volume) of RU 43044, which can be achieved by the jet milling techniques described above, is about 2-3 μm (d50), 10-12 μm (d99) and <15 μm (d100) by using Laser diffractometry.

A preferred dosage form is a multiparticulate system, consisting of multiparticulates of a size of 1-3 mm, which ensures rapid gastric emptying, low variability in gastric transit time, and optimized compound absorption.

Step 1. Preparation of uncoated pellets containing RU 43044 (Formula I). Thirty grams (g) of micronized RU 43044 (Formula I) are added to a mixture of 60 grams microcrystalline cellulose (Avicel® PH-101) and 8 grams hydroxypropyl methylcellulose (HPMC E6). After homogenization of the dry powders, the mixture is granulated by adding an aqueous PVP (25.000 MW) solution (5%, w/w) q.s. The wet granulate obtained is extruded at room temperature at 25 rpm using a CLS Extruder 20 (2 mm orifice diameter) followed by spheronization of the extrudates using a CLS MBS 120 spheronizer with friction plate. The resulting spherical pellets are dried to a humidity of 3-5% containing about 30% (w/w) micronized RU 43044 (Formula I).

Step 2. Preparation of coated pellets for immediate release containing micronized RU 43044 (Formula I). A spray suspension of 100 ml containing 6.25 grams of amino methacrylate copolymer-NF (Eudragit® E 100), 0.625 polyethylene glycol (PEG 6000) and 3.1 grams (g) talcum, dissolved/suspended in acetone/isopropanol 1:1 is prepared by using a shear mixer. The spray suspension is finally passed through a 0.5 mm sieve.

The suspension obtained is sprayed onto 50 grams pellets according to Step 1 using a Caleva lab coater MCD 2. The process conditions are set to: spray rate 0.2-0.3 g/min, inlet temperature 40°−45° C., flow rate 60 L/min at 2 bar, air temperature 30°-35° C. The spraying is maintained up to a 5% increase by weight due to the coating applied. The pellets are finally cured for about one hour at the air temperature of 30°-35° C.

### Example 4

### Preparation of Layered Pellets for Delayed or Pulsatile Release (of Micronized RU 43044 (Formula I))

A spray suspension containing 5.25 grams of ammonio methacrylate copolymer (Eudragit® RL PO Type A), 17.25 grams of ammonio methacrylate copolymer (Eudragit® RS PO Typ B), 3.5 grams triethyl citrate (TEC) and 5.25 grams talcum, dissolved/suspended in 300 grams acetone/isopropanol 1:1 is prepared by using a shear mixer. The spray suspension is finally passed through a 0.5 mm sieve.

The suspension obtained is sprayed onto 50 grams (g) pellets according to Step 2 (Example 3) using a Caleva lab coater MCD 2. The process conditions are set to: spray rate 0.2-0.3 g/min, inlet temperature 40°-45° C., flow rate 60 L/min at 2 bar, air temperature 30°-35° C. The spraying is maintained up to a 7% increase by weight due to the coating applied. The pellets are finally cured for about one hour at the air temperature of 30°-35° C.

A modulated permeability of the coated membrane and an osmotic agent incorporated in the core of the multiparticulate particles is contemplated. In order to achieve an appropriate osmotic pressure within the coated multiparticulate, osmogens of acceptable pharmaceutical grade of hydrophilic nature can be applied. These are preferably low molecular weight sugars like fructose, sucrose, mannitol, inorganic salts like sodium phosphates, or organic acids such as citric acid or tartaric acid. Upon penetration of the gastrointestinal fluid through the membrane of the coated dosage after oral administration the dissolution of the osmogen creates an osmotic pressure, which finally leads to a rupture of the polymeric membrane and releases the compound spontaneously and completely.

The compromise of the coating is influenced by the penetration rate of the gastrointestinal fluid that can be modulated by the adding hydrophilic low or large molecular weight compounds to the coating. Such modulators may be physiologically inert, water-soluble polymers, e.g., low molecular weight methylcellulose or hydroxypropyl-methylcellulose (HPMC), sugars, e.g., monosaccharides such as fructose and glucose, disaccharides such as lactose, sucrose, or polysaccharides such as cellulose, amylose and dextran.

In order to achieve appropriate timing of the release pulse, the modulator may be at least 10 percent, at least about 20 weight percent of the multiparticulate, and usually not more than about 50 weight percent, preferably not more than about 40 weight percent. The polymer coating may comprise at least about 5 and not more than 50 weight percent of the multiparticulate.

The exact proportion of modulator and active agent can be determined by formulation design experiments producing different type of multiparticulates of different amounts of modulator. A USP-approved method for dissolution or release test can be used to measure the rate of release (<711>, USP 32 NF 27,2009, Vol. 1, Apparatus 1 with varying nominal capacities from 1 L to 4 L). In order to better mimic the conditions present in the digestive system and, if necessary to test under infinite sink conditions, a flow-through-cell apparatus according to the USP (Apparatus 4) can be used alternatively to determine the dissolution rate. The detection of the dissolved compound as a function of time may be followed by various state of the art methods, such as spectrophotometrically, HPLC, mass spectroscopy, etc. until the absorbance becomes constant or until greater than 90% of the compound has been released.

### Example 5

### Evaluation of Compound Release Profile for Coated Pellets for Immediate Release RU 43044 (Formula I)

A USP apparatus according to the well described basket method (Sotax AT) is used to determine the in vitro release rate of RU 43044 (Formula I) from the pellets manufactured according to Example 3, Step 2. The dissolution medium is 900 ml of deionized water. The temperature is set and controlled during the test at 37° C. The rotation speed of the basket is set to 50 rpm and kept constant. The unit dosage of pellets is placed in the dry basket at the beginning of the test.

Samples from the dissolution medium in order to determine the concentration of RU 43044 are taken at 5, 10, 20, 30, 45, 60 and 90 minutes, and filtered through Distek 10 μm PE filter. A conventional HPLC apparatus equipped with an UV spectrometer at an absorption of 245 nm is used to measure the concentration of RU 43044 in the samples. There is an immediate release of the compound from the dosage form. After 45 minutes more than 80% of the dose is released, after 90 minutes the compound is nearly completely released and dissolved in the dissolution medium. The amount of compound released and the time—profile of release is influenced by the amount of coating, the amount of talcum, the curing time and the stirring rate of the basket.

### Example 6

### Evaluation of Compound Release Profile for Layered Pellets for Delayed or Pulsatile Release RU 43044 (Formula I)

The unit dosage of pellets from Example 4 is placed in the dry basket at the beginning of the test. The test conditions with regard to rotation speed, temperature and dissolution medium are the same as in Example 5. Samples from the dissolution medium in order to determine the concentration of RU 43044 (Formula I) are taken at 30, 60, 90, 120, 150, 180 and 240 minutes, and filtered through Distek 10 μm PE filter. A conventional HPLC apparatus equipped with an UV spectrometer at an absorption of 245 nm is used to measure the concentration of RU 43044 (Formula I) in the samples. There is a delayed release of the compound from the dosage form. After 60 (90) minutes not more than 10% (15%) of the dose is released, after 120 minutes not more than 20% of the compound is released, after 150 minutes more than 80% of the compound is released and after 180 minutes the release is nearly complete. The amount of compound released and the time-profile of release is influenced by the amount of coating, the amount of talcum and the curing time and the stirring rate of the basket.

The invention is not limited to the embodiments described and exemplified above, but is capable of variation and modification within the scope of the appended claims.

### REFERENCES

1. de Kloet E R; Joels, M.; Holsboer F Stress and the brain: from adaptation to disease. Nature reviews neuroscience 2005.
2. Sapolsky, R. M.; Romero, M. L.; Munck, A. U. How do glucocorticoids influence stress responses? Integrating permissive, suppressive, stimulatory, and preparative actions. Endocrine reviews 2000, 21 (1), 55-89.
3. Tsigos, C.; Kyrou, I.; Kassi, E.; Chrousos, G. P. Stress, endocrine physiology and pathophysiology. 2016.
4. Thorsley D; Leproult R; Spiegel K; Reifman J. A phenomenological model for circadian and sleep allostatic modulation of plasma cortisol concentration. American Journal of Physiology-Endocrinology and Metabolism 2012, 303 (10).
5. Bellavance, M.; Rivest S The HPA-Immune Axis and the Immunomodulatory Actions of Glucocorticoids in the Brain. Frontiers in Immunology 2014, 5.
6. McMaster, A.; Jangani, M.; Sommer, P.; Han, N.; PLoS-áGç^{a}, B. Ultradian cortisol pulsatility encodes a distinct, biologically important signal. PLoS-áGç^{a} 2011.
7. Martocchia, A.; Curto, M.; Toussan, L.; Stefanelli, M.; Falaschi, P. Pharmacological strategies against glucocorticoid-mediated brain damage during chronic disorders. Recent patents on CNS drug discovery 2011, 6 (3), 196-204.
8. Strain, J. J.; and anxiety, F. Considering adjustment disorders as stress response syndromes for DSM-5. Depression and anxiety 2011.
9. Wiemers, U. S.; Schoofs, D.; Stress, W. A friendly version of the Trier Social Stress Test does not activate the HPA axis in healthy men and women. Stress 2013.
10. Vogel, S.; npj of Learning, S. Learning and memory under stress: implications for the classroom. npj Science of Learning 2016.
11. Borbely, A. A.; Daan, S.; Wirz-Justice A; Deboer T The two-process model of sleep regulation: a reappraisal. Journal of Sleep Res 2016.
12. Alexandros, N., V; Julio, F.; Duanping, L.; Edward, O. B. Insomnia with objective short sleep duration: The most biologically severe phenotype of the disorder. Sleep Medicine Reviews 2013, 17 (4), 241-254.
13. Caufriez A; Moreno R; Leproult R; Vertongen F; Van Cauter E; Copinschi G Immediate effects of an 8-h advance shift of the rest-activity cycle on 24-h profiles of cortisol. American Journal of Physiology-Endocrinology and Metabolism 2002, 282 (5).
14. Kwangwook, C. Chronic ‘jet lag’ produces temporal lobe atrophy and spatial cognitive deficits. Nature Neuroscience 2001, 4 (6).

1. A method of preventing, relieving or treating symptoms, disorders and diseases associated with acute stress or temporary hypercortisolism, comprising administering to a subject in need thereof a SASGRM in an amount effective to prevent, relieve or treat the symptoms, disorders and diseases associated with acute stress or temporary hypercortisolism in the subject.

2. The method of claim 1, wherein the SASGRM is selected from hydroxy-androsta-4,9(11)-dien-3-ones, 21-hydroxy-6,19-oxidoprogesterones, 16-hydroxy-11-(substituted phenyl)-estra-4,9-dienes, 17-beta-carboxamides of dexamethasone and Δ1-11-oxa-11-deoxycortisols.

3. The method of claim 1, wherein the SASGRM is selected from a compound of: 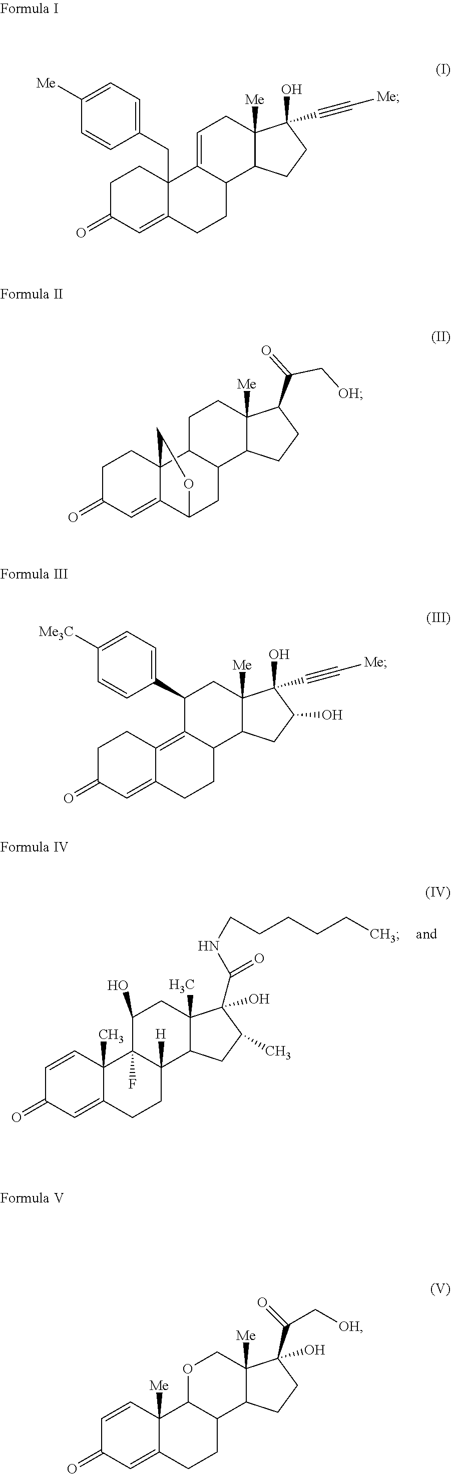
or a combination thereof and pharmaceutically acceptable salts and solvates thereof.

4. The method of claim 1, wherein the symptoms, disorders and diseases associated with acute stress or temporary hypercortisolism are selected from academic problems, educational problems, adjustment disorders, stress-related acute insomnia, chronic insomnia associated with hypercortisolism, chronic insomnia associated with hypercortisolism with objective short sleep duration, circadian rhythm disorder, circadian rhythm disorder of the jet lag type or circadian rhythm disorder of the shift work type.

5. The method of claim 1, wherein the SASGRM comprises a compound of Formula I 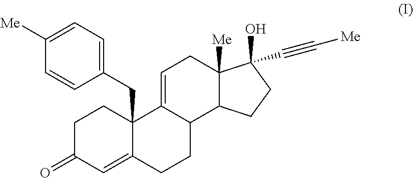 [SMILES: CC#C[C@]1(O)CCC2C3CCC4=CC(=O)CC[C@]4(Cc4ccc(C)cc4)C3=CC[C@@]21C]
or a pharmaceutically acceptable salt or solvate thereof.

6. The method of claim 1, wherein the SASGRM comprises a compound of Formula II 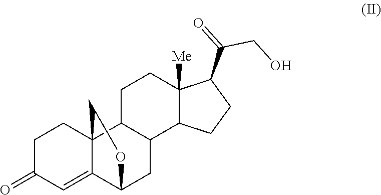 [SMILES: C[C@]12CCC3C(C[C@H]4OC[C@@]35CCC(=O)C=C45)C1CC[C@@H]2C(=O)CO]
or a pharmaceutically acceptable salt or solvate thereof.

7. The method of claim 1, wherein the SASGRM comprises a compound of Formula III 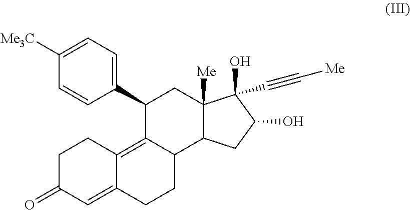
or a pharmaceutically acceptable salt or solvate thereof.

8. The method of claim 1, wherein the SASGRM comprises a compound of Formula IV 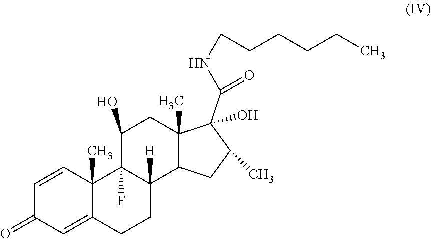
or a pharmaceutically acceptable salt or solvate thereof.

9. The method of claim 1, wherein the SASGRM comprises a compound of Formula V 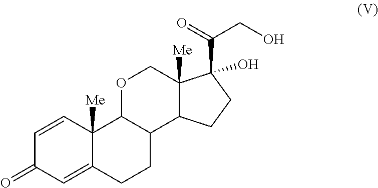 [SMILES: C[C@]12C=CC(=O)C=C1CCC1C2OC[C@@]2(C)C1CC[C@]2(O)C(=O)CO]
or a pharmaceutically acceptable salt or solvate thereof.

10. The method of claim 1, wherein the SASGRM is comprised in a composition comprising a plurality of particles.

11. The method of claim 10, wherein the plurality of particles comprise immediate release particles, delayed release particles, pulsatile release particles or a combination thereof.

12. The method of claim 11, wherein the plurality of particles comprise pulsatile release particles.

13. The method of claim 12, wherein the pulsatile release particles comprise a first population of pulsatile release particles that release the SASGRM following a lag period after the SASGRM is released from the immediate release particles, and a second population of pulsatile release particles that release the SASGRM following a lag period after the SASGRM is released from the first population of pulsatile release particles.

14. The method of claim 13, wherein the pulsatile release particles further comprise a third population of pulsatile release particles that release the SASGRM following a lag period after the SASGRM is released from the second population of pulsatile release particles.

15. The method of claim 11, wherein the delayed release particles and pulsatile release particles comprise a coating.

16. The method of claim 15, wherein the coating comprises a water soluble polymer.

17. The method of claim 16, wherein the water soluble polymer is selected from methylcellulose, hydroxyethyl cellulose, hydroxypropyl cellulose, hydroxypropyl methylcellulose, cellulose acetate phthalate, polyvinylpyrrolidone, polyvinylpyrrolidone/vinyl acetate copolymer, polyvinyl alcohol, polyethylene glycol, polyethylene oxide, hyaluronic acid, alginate, carrageenan, gelatin, and any combination thereof.

18. The method of claim 15, wherein the coating comprises a water insoluble polymer.

19. The method of claim 18 wherein water insoluble polymer is selected from the group consisting of polyvinyl acetate, cellulose acetate, methyl cellulose, ethylcellulose, cellulose acetate butyrate, cellulose acetate propionate, noncrystalline cellulose, polyethylenes, and polyvinyl alcohol, polyacrylates, methacrylates, Eudragit® RS, Eudragit® RL, Eudragit® RS30D, Eudragit® RL30D, a Eudragit® NE30D, Methocel® K100M, Methocel® K15M and any combination thereof.

20. The method of claim 15, wherein the coating further comprises a plasticizer.

21. The method of claim 15, wherein the coating further comprises a glidant.

22. The method of claim 21, wherein the glidant comprises talc.

23. The method of claim 11, wherein the immediate release particles comprise a seal coating.

24. The method of claim 11, wherein the delayed release particles and pulsatile release particles comprise a seal coating.

25. The method of claim 13, wherein the first population of pulsatile release particles releases the SASGRM following a lag period of about 0.5-3 hours after the SASGRM is released from the immediate release particles, and the second population of pulsatile release particles releases the SASGRM following a lag period of about 0.5-3 hours after the SASGRM is released from the first population of pulsatile release particles.

26. The method of claim 25, wherein the first population of pulsatile release particles releases the SASGRM following a lag period of about 1-2 hours after the SASGRM is released from the immediate release particles, and the second population of pulsatile release particles releases the SASGRM following a lag period of about 1-2 hours after the SASGRM is released from the first population of pulsatile release particles.

27. The method of claim 14, wherein the third population of pulsatile release particles releases the SASGRM following a lag period of about 1-3 hours after the SASGRM is released from the second population of pulsatile release particles.

28. The method of claim 27, wherein the third population of pulsatile release particles releases the SASGRM following a lag period of about 1-2 hours after the SASGRM is released from the second population of pulsatile release particles.

29. The method of claim 11, wherein the plurality of particles are comprised in a tablet.

30. The method of claim 11, wherein the plurality of particles are comprised in a capsule.

31. The method of claim 1, wherein the subject is a human having symptoms, disorders or diseases associated with acute stress or temporary hypercortisolism.

32. A composition comprising a plurality of particles comprising immediate release particles, delayed release particles or pulsatile release particles comprising an amount of a SASGRM.

33. The composition of claim 32 wherein the SASGRM is selected from hydroxy-androsta-4,9(11)-dien-3-ones, 21-hydroxy-6,19-oxidoprogesterones, 16-hydroxy-11-(substituted phenyl)-estra-4,9-dienes, 17-beta-carboxamides of dexamethasone, and Δ1-11-oxa-11-deoxycortisols.

34. The composition of claim 32, wherein the SASGRM is selected from a compound of: 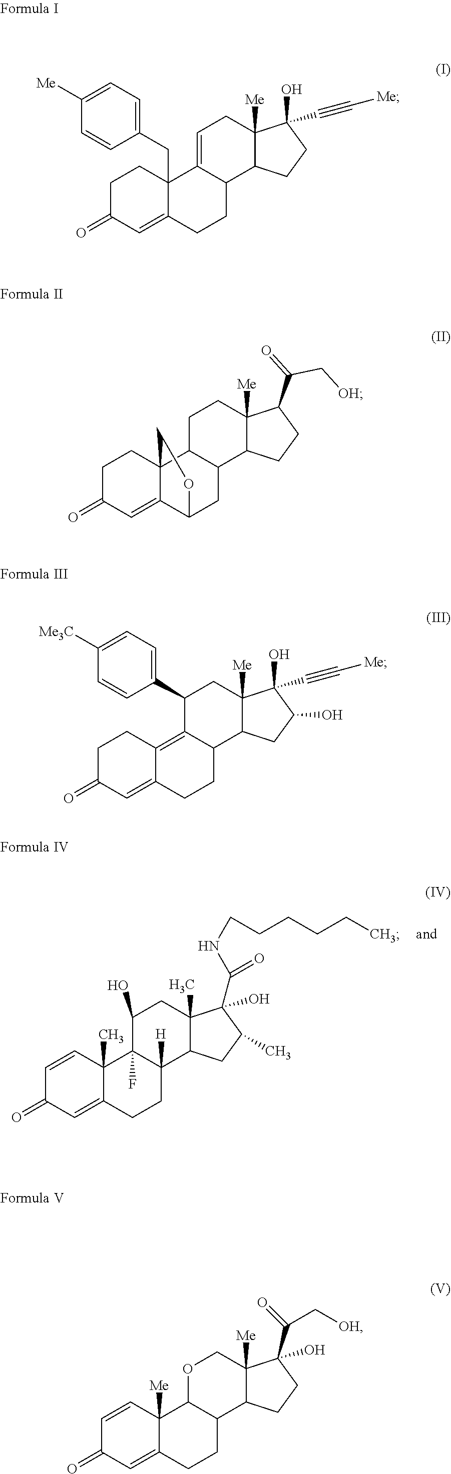
or a combination thereof and pharmaceutically acceptable salts or solvates thereof.

35. The composition of claim 34, wherein the plurality of particles comprise pulsatile release particles.

36. The composition of claim 35, wherein the pulsatile release particles comprise a first population of pulsatile release particles that release the SASGRM following a lag period after the SASGRM is released from the immediate release particles, and a second population of pulsatile release particles that release the SASGRM following a lag period after the SASGRM is released from the first population of pulsatile release particles.

37. The composition of claim 36, wherein the pulsatile release particles further comprise a third population of pulsatile release particles that release the SASGRM following a lag period after the SASGRM is released from the second population of pulsatile release particles.

38. The composition of claim 32, wherein the delayed release particles and pulsatile release particles comprise a coating.

39. The composition of claim 38, wherein the coating comprises a water soluble polymer.

40. The composition of claim 39, wherein the water soluble polymer is selected from the group consisting of methylcellulose, hydroxyethyl cellulose, hydroxypropyl cellulose, hydroxypropyl methylcellulose, cellulose acetate phthalate, polyvinylpyrrolidone, polyvinylpyrrolidone/vinyl acetate copolymer, polyvinyl alcohol, polyethylene glycol, polyethylene oxide, hyaluronic acid, alginate, carrageenan, gelatin, and any combination thereof.

41. The composition of claim 38, wherein the coating comprises a water insoluble polymer.

42. The composition of claim 41, wherein water insoluble polymer is selected from the group consisting of polyvinyl acetate, cellulose acetate, methyl cellulose, ethylcellulose, cellulose acetate butyrate, cellulose acetate propionate, noncrystalline cellulose, polyethylenes, and polyvinyl alcohol, polyacrylates, methacrylates, Eudragit® RS, Eudragit® RL, Eudragit® RS30D, Eudragit® RL30D, a Eudragit® NE30D, Methocel® K100M, Methocel® K15M and any combination thereof.

43. The composition of claim 38, wherein the coating further comprises a plasticizer.

44. The composition of claim 38, wherein the coating further comprises a glidant.

45. The composition of claim 44, wherein the glidant comprises talc.

46. The composition of claim 32, wherein the immediate release particles comprise a seal coating.

47. The composition of claim 32, wherein the delayed release particles and pulsatile release particles comprise a seal coating.

48. The composition of claim 36, wherein the first population of pulsatile release particles releases the SASGRM following a lag period of about 1-3 hours after the SASGRM is released from the immediate release particles, and the second population of pulsatile release particles releases the SASGRM following a lag period of about 1-3 hours after the SASGRM is released from the first population of pulsatile release particles.

49. The composition of claim 48, wherein the first population of pulsatile release particles releases the SASGRM following a lag period of about 1-2 hours after the SASGRM is released from the immediate release particles, and the second population of pulsatile release particles releases the SASGRM following a lag period of about 1-2 hours after the SASGRM is released from the first population of pulsatile release particles.

50. The composition of claim 37, wherein the third population of pulsatile release particles releases the SASGRM following a lag period of about 1-3 hours after the SASGRM is released from the second population of pulsatile release particles.

51. The composition of claim 50, wherein the third population of pulsatile release particles releases the SASGRM following a lag period of about 1-2 hours after the SASGRM is released from the second population of pulsatile release particles.

52. The composition of claim 32, wherein the plurality of particles are comprised in a tablet.

53. The composition of claim 32, wherein the plurality of particles are comprised in a capsule.

54. The composition of claim 32, wherein the composition comprises an effective amount of pulsatile release particles for preventing, relieving or treating symptoms, disorders and diseases associated with acute stress or temporary hypercortisolism.

55. A composition of claim 32 comprising a compound of Formula I 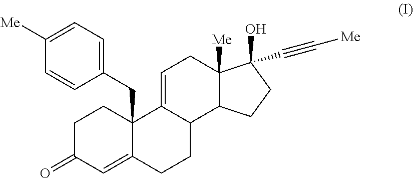 [SMILES: CC#C[C@]1(O)CCC2C3CCC4=CC(=O)CC[C@]4(Cc4ccc(C)cc4)C3=CC[C@@]21C]
or a pharmaceutically acceptable salt or solvate thereof.

56. A composition of claim 32 comprising a compound of Formula II 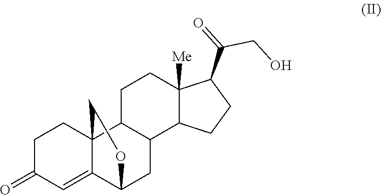 [SMILES: C[C@]12CCC3C(C[C@H]4OC[C@@]35CCC(=O)C=C45)C1CC[C@@H]2C(=O)CO]
or a pharmaceutically acceptable salt or solvate thereof.

57. A composition of claim 32 comprising a compound of Formula III 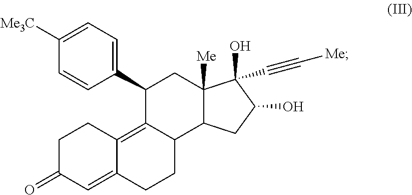
or a pharmaceutically acceptable salt or solvate thereof.

58. A composition of claim 32 comprising a compound of Formula IV 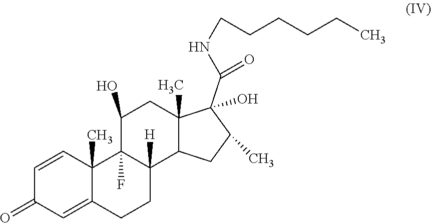
or a pharmaceutically acceptable salt or solvate thereof.

59. A composition of claim 32 comprising a compound of Formula V 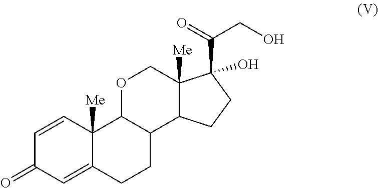 [SMILES: C[C@]12C=CC(=O)C=C1CCC1C2OC[C@@]2(C)C1CC[C@]2(O)C(=O)CO]
or a pharmaceutically acceptable salt or solvate thereof.
